(19) United States
(12) Patent Application Publication
(10) Pub. No.: US 2006/0188533 A1
(43) Pub. Date: Aug. 24, 2006

(54) Delivery of bio-available compounds with anhydrous topical preparations

(76) Inventors: J. Steve Brown, Gilbert, AZ (US); John Hill, Mesa, AZ (US); J. Randall Tryon, Mesa, AZ (US); Michele Ward, Queen Creek, AZ (US)

Correspondence Address: NOBLITT & GILMORE, LLC., 4800 NORTH SCOTTSDALE ROAD, SUITE 6000, SCOTTSDALE, AZ, 85251, US

(21) Appl. No.: 11/340,043

(22) Filed: Jan. 26, 2006

Related U.S. Application Data
(60) Provisional application No. 60/647,677, filed on Jan. 26, 2005.
(60) Provisional application No. 60/670,570, filed on Apr. 11, 2005.

(51) Int. Cl. A61K 36/18; A61K 8/02

(52) U.S. Cl. 424/401; 424/63; 424/725

(57) ABSTRACT

The disclosed systems and methods for delivery of bio-available compounds in topical preparations generally include anhydrous carrier particles adapted to carry bio-available compounds. The carrier particles may be admixed with anhydrous formulation material that is suitably adapted to suspend and/or separate the carrier particles. Disclosed features and specifications may be variously controlled, adapted or otherwise optionally modified to realize improved production and/or use of carrier particles that may be employed to deliver bio-available compounds in order to achieve a desired purpose. Exemplary embodiments of the present invention generally provide anhydrous carrier particles that may be mixed with substantially anhydrous topical preparations.

### RELATED APPLICATIONS

This application claims the benefit of U.S. Provisional Patent Application Ser. No. 60/647,677 filed in the United States Patent and Trademark Office on Jan. 26, 2005 by J. Steve Brown, John Hill, Randy Tryon, and Michele Ward, and U.S. Provisional Patent Application Ser. No. 60/670,570 filed in the United States Patent and Trademark Office on Apr. 11, 2005 by J. Steve Brown, John Hill, Randy Tryon, and Michele Ward.

### FIELD OF INVENTION

The present invention generally relates to cosmetics and bio-available formulations; and more particularly, representative and exemplary embodiments of the present invention generally concern delivery of bio-available compounds in topical preparations.

### BACKGROUND OF INVENTION

Conventional options for providing topical formulations containing bio-available compounds are limited due to unrefined methods of delivery and poor delivery transport kinetics. Additionally, traditional methods do not afford effective substantially anhydrous delivery of bio-formulations that are otherwise unstable in the presence of water, such as Vitamin C (ascorbic acid). Accordingly, stored non-aqueous Vitamin C products typically will have more than 10% potency loss after one year at room temperature in an unopened container.

In the field of cosmetic, personal care, and pharmaceutical products, emollients are usually defined as any agent that softens or smoothes the skin and which tend to reduce roughness, cracking and irritation. In general, smoothing is believed to be affected by the penetration of the emollient material into the surface layers of tissue, by rubbing and massaging action upon application. The ancient Greek physician, Galen, is thought to have made one of the first emollients consisting of beeswax, spermaceti, almond oil, borax and rosewater.

Emollients tend to be bland, fatty, oleaginous substances which may be applied locally to the skin, mucous membranes or abraded tissue. One of the benefits of emollients lies in their ability to exclude water-soluble irritants, air, and air-borne bacteria when a layer of emollient is present. At present, there are numerous materials which function as emollients in a variety of products with a variety of component materials that may act in subtly different ways. For example, certain emollients rest on the surface of the skin and generally serve to impede water loss from the skin. Such component ingredients are generally comprised of large organic molecules that form a hydrophobic barrier to generally prevent water from leaving the surface of the skin. Examples of such emollients are lanolin, mineral oil, silicone derivatives and petroleum jelly.

Emollient substances are commonly employed in cleansing and antiphlogistic creams and lotions. Compound ointment bases, creams and other medical applications are also materials that find use as emollients. Among the more common conventional emollient materials are castor oil, corn oil, cottonseed oil, rose water ointment, apricot kernel oil, avocado oil, grape seed oil, hazelnut oil, olive oil, sesame oil, theobroma oil, almond oil, myristyl alcohol, and recently other natural oils such as jojoba oil.

Other ingredients that have been used as emollients include a number of fatty acids derived from either plant or animal sources. Fatty acids generally comprise aliphatic hydrocarbon or other organic chains with carboxylic substituents thereon, typically having between 8 and 24 carbon atoms in the chain backbone. Fatty acids have been used in creams, lotions, shaving creams, lipsticks and as pressing agents in pressed powders and blushes. Fatty acids, which are typically used in cosmetics formulations, generally include at least one of stearic acid, oleic acid, myristic acid and palmitic acid. Other typical fatty acids include linoleic acid, behenic acid and other common fatty acids of the general formula CₙH₍₂ₙ₊₁₎COOH.

Fatty alcohols have been found to be less sticky and less heavy than many other fatty materials (such as fatty acids), and are frequently used to improve the viscosity and stability of lotions and creams. Fatty alcohols also have utility in reactive hair dying and perming products. Examples of fatty alcohols which find use in the field of cosmetics and personal care products are cetyl alcohol, lauryl alcohol, stearyl alcohol and oleyl alcohol.

Additional examples of emollients are fatty esters. One of the qualities of fatty esters is that they generally do not feel as oily to the touch as some other types of emollient fatty ingredients. Examples include isopropyl palmitate, isopropyl myristate and glyceryl stearate.

Another emollient is jojoba oil, which is generally derived from the seed of the species Simmondsia chinensis. Jojoba is a seed oil with excellent skin feel. The oil is composed almost exclusively of wax esters, with little or no triglycerides present. A major portion of the commercial production of jojoba oil is used by the cosmetic industry as an emollient in a variety of products.

### SUMMARY OF THE INVENTION

In a representative aspect, the present invention provides compositions and methods for delivering bio-available compounds in substantially anhydrous formulations. For example, the present invention may provide delivery of vitamin C (ascorbic acid) in a shelf-stable form that the consumer may use for subsequent application, for example, to their skin. The present invention may also provide, for example, delivery of an anti-acne skincare preparation.

Jojoba ester compositions have been found to function well as a dry (e.g., anhydrous) carriers or vehicles for the application of active materials to the skin or hair of consumers. These esters have been found to be useful in pure or blended forms as carriers in the personal care, cosmetic and pharmaceutical fields of use. Jojoba esters may be provided with a range of properties (from the composition of the ester itself or from additives and blended materials) and may be suitably adapted to provide improved feel when used in conjunction with other conventional carriers, vehicles, bases, actives and additives. Upon application and rubbing of the compositions, the jojoba ester based compositions leave the skin feeling soft (which is typical of high quality emollients), yet provide a mildly persistent coating which carries the actives without leaving a wet or oily feel to the skin of the user.

Advantages of the present invention will be set forth in the Detailed Description which follows and may be apparent from the Detailed Description or may be learned by practice of exemplary embodiments of the invention. Still other advantages of the invention may be realized by means of any of the instrumentalities, methods or combinations particularly pointed out in the claims.

### DETAILED DESCRIPTION OF EXEMPLARY EMBODIMENTS

The following representative descriptions of the present invention generally relate to exemplary embodiments and the inventors' conception of the best mode, and are not intended to limit the applicability or configuration of the invention in any way. Rather, the following description is intended to provide convenient illustrations for implementing various embodiments of the invention. As will become apparent, changes may be made in the function and/or arrangement of any of the elements described in the disclosed exemplary embodiments without departing from the spirit and scope of the invention.

Various representative implementations of the present invention may be applied to any system for providing an anhydrous particle for delivery of a bio-available compound. As used herein, the terms “bead”, “particle”, “sphere”, or any combination or variational derivative thereof, are generally intended to include anything that may be regarded as at least being susceptible to characterization as, or generally referring to a discrete formulation component taken either alone or in combination with a carrier base or solution.

A detailed description of an exemplary application, namely a composition and method for delivering bio-available compounds via jojoba ester beads or micro spheres, is provided as a specific enabling disclosure that may be generalized to any application of the disclosed composition and method for delivering bio-available compounds in topical preparations in accordance with various embodiments of the present invention.

In an exemplary and representative embodiment, the carrier particles comprise beads that are generally soft and suitably adapted to rub into the skin while leaving substantially no debris. These beads or micro spheres may be substantially suspended, for example, in an anhydrous composition, such as that of an anhydrous facial mask adapted to deliver the beads proximate to the surface of the user's skin. The composition may be substantially solid at room temperature, and may be provided in various shapes and sizes (but particularly in the form of spheres or beads). In a representative exemplary embodiment, the carrier particles may comprise beads that are mono-sized to a visible diameter on the order of up to about 50 microns to more than about 5,000 microns, and may be colored so as to be substantially visible in product suspension. These carrier particles, according to various aspects of the present invention, may be produced from combinations of fatty alcohols, isopropyl esters and wax esters obtained from the oil contained in the seed of the jojoba plant (Simmondsia chinensis).

Exemplary emollient delivery compositions generally operate to preserve the excellent skin feel attributed to jojoba oil. The disclosed compositions, in accordance with various representative embodiments of the present invention, also increase the range of applications for cosmetic compositions requiring the use of an emollient that is generally more polar and hydrophilic than that found in naturally occurring jojoba oils (e.g., jojoba wax esters).

Representative compositions generally form stable emulsions much more readily than naturally occurring jojoba oil. Compositions in accordance with various representative aspects of the present invention may also provide excellent emolliency to normally dry cosmetic systems involving high levels of pigments, with the emollient acting as a pigment wetting agent as well as an aid for providing a smooth and even application of the dry system. Various disclosed exemplary formulations may also function as excipients in pressed powder.

Representative compositions comprising fatty alcohols, isopropyl esters and jojoba wax esters (jojoba oil) may be obtained by the base catalyzed alcoholysis reaction between jojoba oil and an alkyl alcohol. In the alcoholysis reaction, examples of the base catalyst materials include, but are not necessarily limited to, metal alkoxides, alkali metal alkoxides, inorganic hydroxides, alkali metal hydroxides, and the like; such as, for example, NaOCH₃ (sodium methoxide), NaOCH₂CH₃ (sodium ethoxide), as well as potassium, calcium and lithium counterparts, KOH, and NaOH (e.g., anhydrous alkali metal hydroxides in alcohol solution, with the alcohol of the solution generally employed as the alcohol used in the reaction). See, for example, U.S. Pat. No. 6,280,746 for a detailed description of representative chemical processes that may be employed to obtain suitably adapted jojoba ester carrier compositions.

In general, bio-available compounds may be loaded into particles (beads) comprising jojoba esters. In an exemplary application, a facial mask may comprise a solution having a bio-available component suspended substantially evenly throughout, or the beads may be substantially separate from the solution. The impregnated beads are generally soft to the touch and may be suitably adapted to substantially disintegrate, rupture, burst or otherwise release bio-available components upon mechanical shear forces or agitation of the mask material by the user; thus liberating the bio-available compounds to the surface of the user's skin. Upon liberation, the bio-available compounds may mix with the material of the mask to spread the bio-available material substantially evenly over the surface of the skin. In a representative exemplary embodiment in accordance with the instant invention, beads formulated from dermophilic material(s) (i.e., having an affinity with skin) may be used, allowing bio-available compounds to migrate closer to the skin cell wall, thereby increasing the likelihood of absorption into the skin. The user may then hydrate the mask as they rinse, thereby disassociating (e.g., activating) bio-available components for absorption into the skin cells.

Representative bio-available compounds that may be delivered via carrier particles, consistent with various exemplary embodiments of the present invention, may include, for example: dermatological active agents, nutritional supplements, cosmetically active ingredients, pharmaceutical preparations, and/or the like.

In an exemplary and representative embodiment of the present invention, an anhydrous exfoliating mask may be prepared with colored, visible jojoba ester beads with a bio-available traffic compound comprising, for example, ascorbic acid (i.e., vitamin C) in a concentration of up to about 0.1% to more than about 50%. The mask may comprise any facial preparation that is anhydrous, generally requires rubbing, followed by hydration (e.g., activation and/or rinsing).

The particle beads are generally soft and suitably adapted to rub into the skin while generally leaving no debris upon mechanical abrasion, and may be further adapted to carry other bio-available components in addition to ascorbic acid. In a representative exemplary embodiment, the particles may be substantially mono-sized to a visible diameter of up to about 100 microns to more than about 5,000 microns. The carrier particles may also be colored to impart an aesthetic appeal to the product formulation for the user.

Coloring and fragrancing of the carrier particles may be commercial desirable, but should not be construed as a required or essential feature of the invention. If the carrier particles are adapted to provide a color or tint during application, then this may aid the user by providing a visual cue corresponding to when a suitable amount of mechanical rubbing has occurred and in which application sites. An un-pigmented bead would generally be colorless (e.g., “water white”). This may be desirable in various types of products.

Other bio-available compound formulations which degrade in the presence of water may be desirable, but should not be construed as a required or essential feature of the present invention. Alternatively, conjunctively or sequentially, various other bio-available compounds may be employed in place of or in addition to, for example, ascorbic acid. For example, in another representative and exemplary embodiment of the present invention, dermatological active agents, such as anti-acne agents, may be delivered via carrier particles in an anhydrous facial mask formulation to freshly exfoliated skin. As the user applies an exfoliating mask to remove dead skin cells from the surface of the skin, particles may thereafter introduce anti-acne active agents to the freshly exfoliated sites via rupture or other known mechanisms of delivery of active agents.

According to various aspects of the present invention, an anhydrous facial mask may be further adapted to provide a substantially “self-heating” function to bring the formulation to a suitable temperature upon application or hydration and/or to signify that cleaning activation has occurred or that a given period of time has elapsed (such as a timer function), indicating that the mask may be subsequently rinsed from the user's skin. If the mask is self-heating, then bio-available absorption may be more efficient due to increased kinetic transport at the skin cell wall. Any mask formulation, whether now known or subsequently hereafter described in the art, may be employed to achieve a substantially similar result as that of the instant invention.

A representative moisturizing warming mask may be obtained by the following procedure:

1. Premix FLORAESTERS K-100 in PEG-8 of a first phase at substantially room temperature.

2. Allow time for the K-100 to substantially completely dissolve into the solution of the first phase.

3. Slowly sift KLUCEL into PEG-8 of a second phase at substantially room temperature with moderate propeller agitation.

4. Allow time for the second phase to substantially completely mix.

5. After approximately 30 minutes of mixing, add the first phase to the second phase and raise the temperature of the combined solution to approximately 75-80 degrees Celsius.

6. Add any remaining ingredients to the mixture with slow to moderate homomixer agitation for approximately 30 minutes.

7. Allow the mix to substantially completely cool (e.g., 3 hours) to room temperature.

8. Add FLORASOMES (a representative carrier particle for bio-available components; see, e.g., U.S. Pat. No. 6,280,746) at room temperature with careful mixing until substantially uniform dispersion is achieved.

A representative component ingredient list for the above-described exemplary mask formulation may include, for example: a first phase comprising polyglycol E-400 (PEG-8 ) available from Dow Chemical (approximately 20% by combined weight), and FLORAESTERS K-100 Jojoba (hydrolyzed jojoba esters and/or jojoba esters and/or water) available from International Flora Technologies, Ltd., Chandler, Ariz., USA (approximately 1.5% by weight); a second phase comprising polyglycol E-400 (PEG-8 ) available from Dow Chemical (Q.S.), KLUCEL LF (hydroxypropyl cellulose) available from Hercules (approximately 3% by weight), PLURACARE F127P (POLOXAMER 407) available from BASF (approximately 2% by weight), KAOLIN 2747 available from Whittaker, Clark & Daniels (approximately 5% by weight), titanium dioxide 9985AW available from LCW (approximately 1% by weight), magnesium sulfate (anhydrous) available from Amresco (approximately 20% by weight), ZEORUM A-5 (zeolite) available from Ikeda (approximately 6% by weight), and preservative(s) (Q.S.); and a third phase component comprising, for example, FLORASOMES Jojoba (jojoba esters) available from International Flora Technologies, Ltd., Chandler, Ariz., USA (approximately 2.25% by weight).

FLORAESTERS K-100 Jojoba has been demonstrated to provide a substantive, pleasant and smooth skin feel in representative mask formulations. FLORASOMES may be suitably adapted to provide, for example, fragrance and/or color signals to the user to enhance the aesthetic appeal of the product. Magnesium sulfate generally soothes and softens rough skin and unclogs pores to reduce the appearance of blemishes. In a representative and exemplary application, FLORASOMES JOJOBA MXS 10% CITRUS FRAGRANCE TOPAZ and/or FLORASOMES JOJOBA SMS 10% CELLINI BLUE NATURAL may be used. Also in a preferred representative and exemplary embodiment, the particle size of magnesium sulfate may be up to about more than about 50 microns to achieve a smooth skin-feel.

Although the specifically enabling embodiments disclosed herein suggest the use of jojoba ester beads as the delivery vehicle for improving the availability of bio-available compounds, any anhydrous particle formulation, whether now known or otherwise hereafter described in the art, may be alternatively, conjunctively or sequentially employed; including for example: ethylhexyl palmitate, petrolatum (VASELINE), polyethylene, and/or the like.

Other bio-available compounds that may be delivered via a substantially anhydrous particle formulation, consistent with various exemplary embodiments of the present invention, may include, for example:

Dermatological active agents, including, for example: benzoyl peroxide, ozonides, retinoids, salicylic acid, and/or the like;

Nutritional supplements;

Cosmetically active ingredients;

Pharmaceutical preparations;

Fruit and/or vegetable extracts and/or juices, including, for example: apple, apricot, asparagus, beet, black currant, blackberries, boysenberry, broccoli, cabbage, carrot, celery, cherry, cranberry, red currant, elderberry, garlic, gooseberry, grape, grapefruit, lemon, lettuce, lime, loganberry, mustard, onion, orange, parsley, passion fruit, pea, peach, pear, pineapple, plum, prune, quince, raspberry, rhubarb, spinach, squash, strawberry, tangerine, tomato, turnip, watercress, and/or the like;

Alpha and beta hydroxy acids, such as, for example: glycolic acid, lactic acid, malic acid, oxalic acid, salicylic acid, tartaric acid, and/or the like;

Hyaluronic acid;

Natural antibiotics, such as, for example: mycosubtilin, actidione, nisin, pimaricin, microsubtilin, patuline, and/or the like;

Vitamins, such as, for example: vitamin B₁ (thiamin), vitamin B₂ (riboflavin), niacin (nicotinic acid, nicotinamide, vitamin PP), vitamin H (biotin), vitamin B₆, vitamin B₁₂, and/or the like;

Amino acids, such as, for example: alanine, valine, leucine, tyrosine, glutamic acid, tryptophan, methionine, lysine, iosleucine, phenylalanine, glycine, cystine, aspartic acid, histidine, arginine, ornithine, serine, asparagines, praline, aminobutyric acid, threonine, and/or the like;

a protein, a peptide, a peptide combination, and/or the like;

Polar extracts of fragrance materials, such as, for example: agrumen oil, allium oil, ambergris, ambrette seed, amyris oil, angelica root, angelica seed, anise, aniseed oil, Artemisia oil, balm mint, balsam fir oil, basil oil, bay oil, bergamot oil, birch tar oil, bitter almond oil, bois de rose oil, buchu leaf oil, cabreuva oil, calamus oil, camphor, cananga oil, caraway oil, cardamom oil, carrot seed oil, cassia oil, castoreum, cedar leaf oil, cedar oil, celery seed oil, chamomile oil, cinnamon bark oil, cistus oil, citronella oil, civet, clary sage oil, clove oil, cognac oil, cola, copaiba oil, coriander oil, cornmint oil, costus root oil, cumin oil, cypress oil, dill seed oil, dill weed oil, elemi oil, estragon oil, eucalyptus oil, eugenol, fennel oil, fir needle oil, galbanum oil, garlic oil, geranium oil, ginger grass oil, ginger oil, grapefruit oil, green cognac oil, guiaiac wood oil, gurjun balsam oil, helichrysum oil, hemlock oil, hiba oil, jasmine oil, juniper berry oil, labdanum oil, laurel leaf oil, lavandin oil, lavandula oil, lavender oil, leek oil, lemon grass oil, lime oil, linaloe oil, litsea cubeba oil, lovage oil, mandarin oil, marjoram oil, menthe arvensis oil, menthe piperita oil, musk, musk ambrette, myrrh oil, nerol, neroli oil, nutmeg oil, oakmoss oil, olibanum oil, onion oil, opopanax oil, orange oil, origanum oil, orris root oil, palmarosa oil, parsley herb oil, patchouli oil, pennyroyal oil, rose oil, rosewood oil, rue oil, sage oil, sandalwood oil, sassafras oil, savory oil silver fir oil, spearmint oil, spike lavender oil, spruce oil, star anise oil, tarragon oil, tea tree oil, terpineols, thujopsene, thyme oil, tolu balsam oil, valerian oil, vanilla oil, verbena oil, vetiver oil, violet leaf alcohol, wood turpentine oil, ylang-ylang oil, and/or the like;

Polar extracts or juices of botanicals, such as, for example: acacia, abies alba, abietin, absinth, acacia decurrens (mimosa), acacia excelsa (bois de rose), aleppy cardamom, allsprice, aloe vera (aloe barbadensis), aloe perei, algae, anhydrol mate, anhydrol tea, apiole (parsley), apium graveolens (celery seed), arrowroot, armoracia lapathifolia (horseradish), arnica, asarum canadense (snakeroot), ascophyllum, aspergillus oryzae (soybean), azulene (chamomile), banana, bamboo, bay laurel, betula alba (birch bud), birch, birch leaf, blazing star (deertongue), black walnut, bladderwrack, boronia, boswellia (olibanum), bruyere, buchu, burdock, butterfly lily, cade, cajuput, calamus, calendula officialis (marigold), capsicum, clove, coltsfoot, comfrey, coneflower, cedar, coconut, coffee, cactus, cedar, chapparal, chickweed, cedarwood, cherry birch, cicely (anise), cistus, citrus aurantium (neroli, orange flower, petitgrain), cucumber, cyclotene (fluove, lovage, maple), cytisus scoparius (broom absolute), dacrydium elatum, dalbergia latifolia (bois de rose), dandelion, daucus (carrot seed), diorissimo (lily of the valley), eagle wood (agar), eau de brouts (orange flower, petitgrain water absolute), elder, elder flower, elemi, estragon, eucalyptus, eucaria spicata (sandalwood), eudesmol (araucaria concrete), evernia furfuracea (oakmoss, treemoss), exaltolide (musk), eyebright, farnesol (ambrefte absolute), fennel, fenugreek, fern, ficus carica, firmoss, flouve ordorante, gardenia, gensing, geranium, ginger, glycyrrhiza glabra (licorice), goldenrod, goldenseal, gorse, guaiacwood, guava, garlic, grape, green tea, hayata (machilus), hemlock spruce, henna, hibiscus abelmoschus (ambrette seed), honey, hop absolute, horse chestnut, horsetail, hyacinth orientalis, hyssop, inchigrass, inula helenium (elecampane), ivy leaf, irish moss, jaborandi, jacmal (curacao peel), jasmine, jonquil, juniper, karma, keora (pandanus), kava-kava, lavender, lemon, lemon bioflavanoids, lemongrass, laminaria, lilac, lime, locus bean, lovage, machilus (hayata), magnolia, majorana hortensis (marjoram), macadamia, mallow, melon, mandarin oil, maple, mastic, mate, matricaria chamomilla (chamomile), mango, may-chang (litsea cubeba), melaleuca, Melissa officianalis, menthe citrate, mistletoe, moschus moschiferus, mugwort, myrrh, myrtle, narcissus jonquilla, nepeta cataria, nettle, nutmeg, oakmoss, oakbark, ocimum gratissimum, olive, opopanax, Oregon fir, origanum hirtum, orris paprika, passionflower, passionfruit, pellitory root, pennyroyal, perilla frutescens, petitgrain cedrat, phellandrene (elemi, angelica, eucalyptus), picea excelsa, pimenta berry, pine, pinetree, piper crassipes, piper longum, pollen, papaya, prunus armeniaca, pulegone (pennyroyal-moroccan), quinine, red clover, roman chamomile, rondeletia (bay leaf, clove bud, lavandin), rosa centifolia, rosa damascene, rose hips, rosemary, rose otto, rhatany, rice, rosewood, rosin, ruta graveolens (rue), saffron, sage clary, St. John's bread (carob), St. John's wort (everlasting absolute), salvia lavandulaefolia (rosemary), salvia hispanica (chia), salvia sclarea (sage clary), sandarac, santalum citrinum, schinus molle, smilax (sasparilla), sesame, sea buckthorn, sea fennel, sea kelp, spirulina, sesame, soap bark, sumbul root, sweet myrtle (calamus), syringe vulgaris (lilac), tagetes glandulifera, tagetes patula, tea tree (melaluca), thea sinensis, thyme, thymus capitatus, tolu balsam, toona calantas, tsuga Canadensis, tuberose, tumeric, ulex europaeus, valerian officinalis, vanilla, verbena, vetiver zizanoides, violet flower, viscum album (mistletoe), wallflower, walnut, wattle, wild cherry, witch hazel, wormseed, wormwood, xanthoxylum alatum, yarrow, ylang-ylang, yucca, zedoaria, zingerone officinale (ginger), and or the like; and

Glitter, special effects pigments, color shifting pigments, and/or the like.

Additional components of the formulation may also include at least one of the following: preservatives, coloring agents, fragrances, essential oils, and/or the like.

In the foregoing specification, the invention has been described with reference to specific exemplary embodiments; however, it will be appreciated that various modifications and changes may be made without departing from the scope of the present invention as set forth herein. The specification is to be regarded in an illustrative manner, rather than a restrictive one and all such modifications are intended to be included within the scope of the present invention. Accordingly, the scope of the invention should be determined by the claims and their legal equivalents rather than by merely the examples described above.

For example, the steps recited in any method or process embodiment may be executed in any order and are not limited to the specific order presented in the claims. Additionally, the components and/or elements recited in any apparatus or composition embodiment may be assembled or otherwise operationally configured in a variety of permutations to produce substantially the same result as the present invention and are accordingly not limited to the specific configuration recited in claims.

Benefits, other advantages and solutions to problems have been described above with regard to particular embodiments; however, any benefit, advantage, solution to problem or any element that may cause any particular benefit, advantage or solution to occur or to become more pronounced are not to be construed as critical, required or essential features or components of the invention.

As used herein, the terms “comprising”, “having”, “including” or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present invention, in addition to those not specifically recited, may be varied or otherwise particularly adapted to specific environments, manufacturing specifications, design parameters or other operating requirements without departing from the general principles of the same.

We claim:

1. A composition suitably adapted to deliver a bio-available compound to a user's skin, said composition comprising:
a substantially anhydrous carrier particle suitably adapted to carry a bio-available compound; and
a substantially anhydrous topical formulation, wherein said topical formulation is suitably adapted to at least one of suspend and substantially separate a plurality of anhydrous carrier particles.

2. The composition of claim 1, wherein said bio-available compound comprises at least one of: a preservative; a dermatological active agent; ascorbic acid; a nutritional supplement; a cosmetically active ingredient; a pharmaceutical preparation; an anti-acne agent; an anti-acne active agent; a fruit juice; a fruit extract; a vegetable juice; a vegetable extract; an alpha hydroxy acid; a beta hydroxy acid; hyaluronic acid; an antibiotic; a natural antibiotic; a vitamin; an amino acid; a protein; a peptide, a peptide combination; a fragrance material; a polar extract of fragrance material; a botanical material; a polar extract of a botanical material; a juice of a botanical material; glitter; a special-effect pigment; a coloring agent; a dye; a color shifting pigment; and an active agent.

3. The composition of claim 1, wherein said carrier particle comprises at least one of: a jojoba fatty alcohol; a jojoba ester; a jojoba isopropyl ester; a jojoba wax ester; ethylhexyl palmitate; petrolatum; polyethylene; a dermophilic material; and an anhydrous material.

4. The composition of claim 1, wherein said carrier particle is substantially configured to at least one of: demonstrate suitable adaptation for rubbing into the skin of the user; demonstrate softness upon application to the skin; leave substantially no debris; maintain substantially uniform shape; substantially comprise a solid at room temperature; maintain substantially uniform size; demonstrate optical properties corresponding to at least one of transparence, translucence and opacity; comprise a diameter of up to about 100 microns to more than about 5000 microns; and demonstrate suitable adaptation for at least one of substantially disintegrating, rupturing, bursting and releasing said bio-available compound upon at least one of application of mechanical shear forces and agitation of said carrier particle.

5. The composition of claim 1, wherein said anhydrous topical formulation is an anhydrous facial mask formulation.

6. The composition of claim 5, wherein said anhydrous topical formulation further comprises a self-heating function.

7. The composition of claim 6, wherein said self-heating function at least one of: creates a substantially smooth skin feel; increases absorption efficiency; increases kinetic transport at the skin cell wall; acts as timer; and indicates that said composition is ready to be rinsed off.

8. The composition of claim 6, wherein said anhydrous topical application further comprises at least one of: a PEG-8; a hydrolyzed jojoba ester; water; hydropropyl cellulose; POLOXAMER 407; KAOLIN 2747; titanium dioxide 9985AW; anhydrous magnesium sulfate; zeolite; a preservative; and a jojoba ester.

9. The composition of claim 8, wherein said composition further comprises at least one of: approximately 20% PEG-8 (wt/wt); approximately 1.5% water (wt/wt); approximately 3% hydroxypropyl cellulose (wt/wt); approximately 2% POLOXAMER 407 (wt/wt); approximately 5% KAOLIN 2747 (wt/wt); approximately 1% titanium dioxide 9985 (wt/wt); approximately 30% anhydrous magnesium sulfate (wt/wt); approximately 6% zeolite (wt/wt); a suitable amount of preservative; and approximately 2.25% of at least one of jojoba esters and hydrolyzed jojoba esters (wt/wt).

10. The composition of claim 1, wherein said bio-available compound comprises a concentration of about at least 0.1% to approximately 50% ascorbic acid (wt/wt of carrier particle).

11. The composition of claim 1, wherein said bio-available compound is at least one of incorporated into and carried by said carrier particle.

12. A method for delivering bio-available compounds to a user's skin, said method comprising the steps of:
providing a substantially anhydrous carrier particle suitably adapted to carry a bio-available compound;
providing a substantially anhydrous topical formulation, wherein said topical formulation is suitably adapted to at least one of suspend and substantially separate a plurality of anhydrous carrier particles.

13. The method of claim 12, wherein said bio-available compound comprises at least one of: a preservative; a dermatological active agent; ascorbic acid; a nutritional supplement; a cosmetically active ingredient; a pharmaceutical preparation; an anti-acne agent; an anti-acne active agent; a fruit juice; a fruit extract; a vegetable juice; a vegetable extract; an alpha hydroxy acid; a beta hydroxy acid; hyaluronic acid; an antibiotic; a natural antibiotic; a vitamin; an amino acid; a protein; a peptide, a peptide combination; a fragrance material; a polar extract of fragrance material; a botanical material; a polar extract of a botanical material; a juice of a botanical material; glitter; a special-effect pigment; a coloring agent; a dye; a color shifting pigment; and an active agent.

14. The method of claim 12, wherein said carrier particle comprises at least one of: a jojoba fatty alcohol; a jojoba ester; a jojoba isopropyl ester; a jojoba wax ester; ethylhexyl palmitate; petrolatum; polyethylene; a dermophilic material; and an anhydrous material.

15. The method of claim 12, wherein said carrier particle is substantially configured to at least one of: demonstrate suitable adaptation for rubbing into the skin of the user; demonstrate softness upon application to the skin; leave substantially no debris; maintain substantially uniform shape; substantially comprise a solid at room temperature; maintain substantially uniform size; demonstrate optical properties corresponding to at least one of transparence, translucence and opacity; comprise a diameter of up to about 100 microns to more than about 5000 microns; and demonstrate suitable adaptation for at least one of substantially disintegrating, rupturing, bursting and releasing said bio-available compound upon at least one of application of mechanical shear forces and agitation of said carrier particle.

16. The method of claim 12, wherein said anhydrous topical formulation is an anhydrous facial mask formulation.

17. The method of claim 16, wherein said anhydrous topical formulation further comprises a self-heating function.

18. The method of claim 17, wherein said self-heating function at least one of: creates a substantially smooth skin feel; increases absorption efficiency; increases kinetic transport at the skin cell wall; acts as timer; and indicates that said composition is ready to be rinsed off.

19. The method of claim 16, wherein said anhydrous topical application further comprises at least one of: a PEG-8; a hydrolyzed jojoba ester; water; hydropropyl cellulose; POLOXAMER 407; KAOLIN 2747; titanium dioxide 9985AW; anhydrous magnesium sulfate; zeolite; a preservative; and a jojoba ester.

20. The method of claim 16, wherein said composition further comprises at least one of: approximately20% PEG-8 (wt/wt); approximately 1.5% water (wt/wt); approximately 3% hydroxypropyl cellulose (wt/wt); approximately 2% POLOXAMER 407(wt/wt); approximately 5% KAOLIN 2747(wt/wt); approximately 1% titanium dioxide 9985 (wt/wt); approximately 30% anhydrous magnesium sulfate (wt/wt); approximately 6% zeolite (wt/wt); a suitable amount of preservative; and approximately 2.25% of at least one of jojoba esters and hydrolyzed jojoba esters (wt/wt).

21. The method of claim 16, further comprising the step of at least one of: premixing jojoba esters in PEG-8; substantially dissolving said jojoba esters into said PEG-8; slowly lifting hydroxypropyl cellulose into PEG-8 with agitation; substantially mixing said hydroxypropyl cellulose with said PEG-8; adding said jojoba esters in said PEG-8 to said hydroxypropyl cellulose with said PEG-8; optionally adding at least one additional ingredient to said composition with agitation; substantially cooling said composition; and adding said carrier particle at room temperature with mixing until substantially uniform dispersion results.

22. The method of claim 12, wherein said bio-available compound comprises a concentration of about at least 0.1% to approximately 50% ascorbic acid (wt/wt of carrier particle).

23. The method of claim 12, wherein said bio-available compound is at least one of incorporated into and carried by said carrier particle.
